(19) United States
(12) Patent Application Publication
(10) Pub. No.: US 2018/0059714 A1
(43) Pub. Date: Mar. 1, 2018

(54) SMART DEVICE WITH DETACHABLE BAND

(71) Applicant: QUALCOMM Incorporated, San Diego, CA (US)

(72) Inventors: Russel Martin, Menlo Park, CA (US); Shashank Narayanan, Sunnyvale, CA (US)

(21) Appl. No.: 15/245,072

(22) Filed: Aug. 23, 2016

(51) Int. Cl. G06F 1/16; A61B 5/00; A61B 5/0205; A61B 5/01

(52) U.S. Cl. CPC G06F 1/163; G06F 1/1635; G06F 1/1688; A61B 5/681; A61B 5/0533; A61B 5/015; A61B 5/742; A61B 5/4806; A61B 5/02055

(57) ABSTRACT

Methods, systems, computer-readable media, and apparatuses for a smart device are presented. In some implementations, a system comprises a main device including a processor, and a band including one or more sensors, memory, and a battery. The band may be configured to communicatively and mechanically connect to the main device, and to store data received from the one or more sensors into the memory. Upon being connected to the band, the processor may be configured to obtain the stored data from the memory of the band, and process the stored data obtained from the memory of the band.

### FIELD

Aspects of the disclosure relate to wearable electronic devices.

### BACKGROUND

Wearable electronic devices, such as, for example a smartwatch or the like, have been increasing in popularity. Besides simply telling the time, a smartwatch may provide other features such as, for example, health tracking, mobile notifications, mobile applications, etc. One such feature, sleep monitoring, has grown in popularity perhaps because the feature may provide for monitoring a user's movement, heart rate, etc., during a period in which a user may be asleep at times. Accordingly, a user may be provided with vital information pertaining to their sleep habits, and the like.

However, a smartwatch typically has limited on-board power resources. As such, in certain instances (e.g., at night time), there may be a need to recharge the batteries in the smartwatch. Recharging a smartwatch whilst worn by a user attempting to sleep may not be practical, for example if a power adapter and cord is used. Further still, some users may find that some smartwatches may be uncomfortable to wear during sleep. Hence, for these potential reasons and/or others, there is need for an improved “smart device”, e.g., a smartwatch or other like wearable electronic device.

### BRIEF SUMMARY

In accordance with certain aspects, a system may be provided which comprises a main device and a band. Here, for example, the main device may comprise a processor and the band may comprise one or more sensors, memory, and a battery. The band and the main device may be configured to be communicatively and mechanically connected to one another. The band may be configured to store data received from the one or more sensors into the memory. The processor, wherein being connected to the band, may be configured to obtain all or part of the stored data from the memory of the band, and to process all or part of the stored data obtained from the memory of the band

In accordance with certain aspects, a method may be provided which includes receiving an indication that a band comprising one or more sensors, memory, and a battery, has been communicatively and mechanically connected to a main device. The method also includes, in response to receiving the indication that the band has been communicatively and mechanically connected to the main device, obtaining data stored in the memory that comprises data received by the band from the one or more sensors, and.

In some implementations, one or more non-transitory computer-readable media storing computer-executable instructions, when executed, cause one or more computing devices included in a mobile device to receive an indication that a band comprising one or more sensors, memory, and a battery, has been communicatively and mechanically connected to a main device, in response to receiving the indication that the band has been communicatively and mechanically connected to the main device, obtain data stored in the memory that comprises data received by the band from the one or more sensors, and process the stored data obtained from the memory.

### BRIEF DESCRIPTION OF THE DRAWINGS

Aspects of the disclosure are illustrated by way of example. In the accompanying figures, like reference numbers indicate similar elements.

FIG. 1 illustrates a simplified diagram of a system that may incorporate one or more implementations;

FIG. 2 illustrates a simplified block diagram of a main device that may incorporate one or more implementations;

FIG. 3 illustrates a simplified block diagram of a band that may incorporate one or more implementations;

FIG. 4 depicts a system 100 including a main device and a band according to some implementations;

FIG. 5 illustrates a main device configured to be communicatively and mechanically connected to a band;

FIG. 6 illustrates a main device communicatively and mechanically connected to a band; and

FIG. 7 illustrates an example of a computing system in which one or more embodiments may be implemented.

FIG. 8 illustrates an example process that may be implemented in a system, for example, as illustrated in FIG. 1, in accordance with certain aspects.

### DETAILED DESCRIPTION

Several illustrative implementations will now be described with respect to the accompanying drawings, which form a part hereof. While particular implementations, in which one or more aspects of the disclosure may be implemented, are described below, other implementations may be used and various modifications may be made without departing from the scope of the disclosure or the spirit of the appended claims.

Certain implementations are described that regarding a “smart device” (referred to here for simplicity as a smartwatch) having a face and a band, where the band is detachable from the face. The band may include its own battery, sensors, memory, and control electronics, or some combination thereof. At night, the user may detach the watch face from the smartwatch and connect the watch face to a charger to charge overnight. The user may continue to wear only the band portion of the smartwatch as he/she retires for the night. The watch band may continue collecting data overnight (using its own battery, sensors, and memory). The next time (e.g., in the morning) the user reconnects the watch band with the watch face, the data collected by the watch band may be transferred to the watch face, where it can be processed, stored, and shared. In some implementations, the sensors within the band can replicate, augment or potentially replace all or parts of certain sensors of the watch face. In some implementations, processing of sensor obtained data can be done within the band itself. At times, the raw data outputted by some sensors may be extremely dense. In such cases, it may be advantageous to process the raw data in the band prior to passing the band-processed data to the main device.

The watch band may contain sensors such as, but not limited to, accelerometer, gyroscope, skin resistance, skin temperature, ambient temperature, ambient light, heart rate, and microphone. Further, the watch band may include a battery that powers the sensors and is configured to operate throughout the night. The watch band battery may be charged by the watch face's larger battery when the watch band is reattached to the watch face. An electronic interface (e.g., Inter-Integrated Circuit (I2C))) may exist between the watch band and the watch face, for transferring data. The I2C interface may also be operable for setting data collection parameters. For example, the I2C interface may be used to set an output data rate of one or more of the sensors located within the band.

In some implementations, a system comprises a main device including a processor, and a band including one or more sensors, memory, and a battery. The band may be configured to communicatively and mechanically connect to the main device, and to store data received from the one or more sensors into the memory. Upon being connected to the band, the processor may be configured to obtain the stored data from the memory of the band, and process the stored data obtained from the memory of the band.

In some implementations, upon being connected to the band, the main device may be further configured to charge the battery of the band.

In some implementations, the main device further comprises a wireless interface, and the processor is further configured to initiate transmission of data to another device via the wireless interface.

In some implementations, the band is configured to communicatively connect to the main device via an Inter-Integrated Circuit (I²C) bus.

In some implementations, the one or more sensors comprises at least one of: an accelerometer, a gyroscope, a galvanic skin response (GSR) sensor, a skin temperature sensor, an ambient temperature sensor, an ambient light sensor, a heart rate monitor, a bioelectric impedance measuring device, a barometer, or a microphone.

In some implementations, the band is further configured to maintain a wireless communicative connection with the main device without being mechanically connected to the main device.

In some implementations, the band is configured to be interchangeable by a replacement band, the replacement band being configured to be communicatively and mechanically connected to the main device.

In some implementations, the processor is further configured to affect at least one of a display, a speaker, a haptic mechanism, or a light source, of the main device in response to at least one of: (1) a determination that the main device is communicatively connected to the band; (2) a determination that the main device is communicatively disconnected from the band; (3) a determination that the main device is mechanically connected to the band; (4) a determination that the main device is mechanically disconnected from the band; (5) a determination that the processor has obtained at least a portion of the stored data from the memory of the band; (6) a determination that the main device has processed at least a portion of the stored data obtained from the memory of the band; (7) a determination corresponding to a state of the battery of the band; or (8) some combination of (1) through (7).

In some implementations, the battery of the band has a charge capacity between 20 milliampere hour (mAh) to 100 mAh.

In some implementations, the data received from the one or more sensors comprises data associated with tracking properties of a user's sleep.

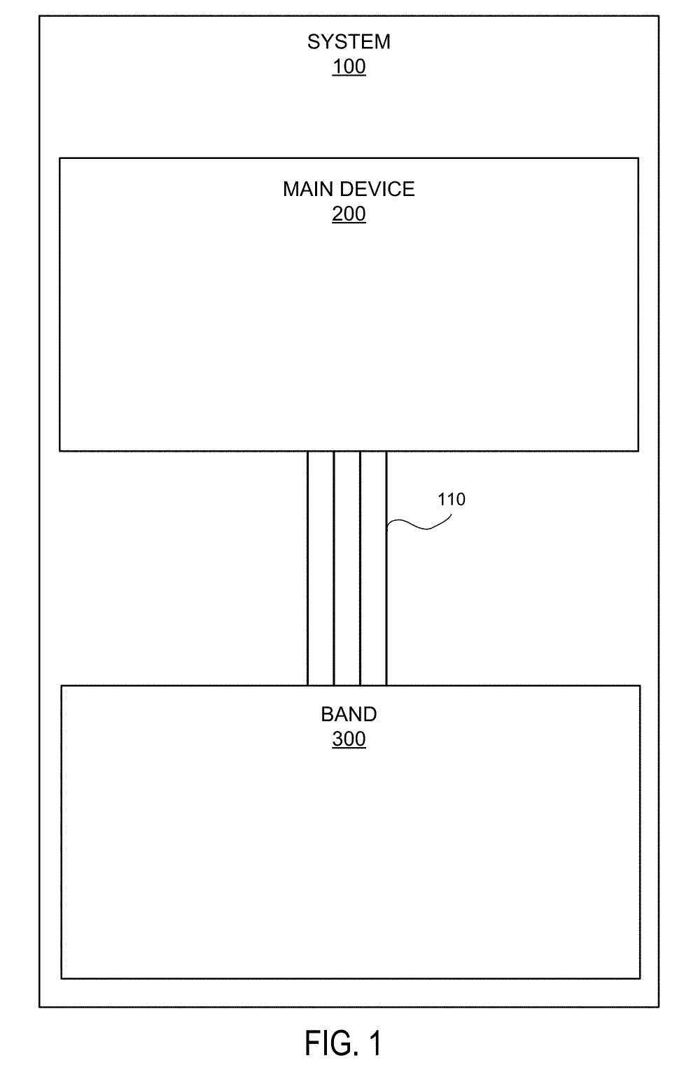

FIG. 1 illustrates a simplified diagram of a system 100 that may incorporate one or more implementations. The system 100 may include a main device 200 and a band 300. The main device 200 and the band 300 may each include one or more mechanical and electrical components. In some implementations, the “system 100” may refer to both the main device 200 and the band 300. The system 100 may also include a bus 110. The bus 110 may operate to facilitate the transfer of data between the main device 200 and the band 300. Bus 110 is intended to represent at least one technology for communicatively connecting main device 200 and band 300. In some implementations, the bus 110 may include an Inter-Integrated Circuit (I2C) bus. In some implementations, the bus 110 may include one or more electrical connections, one or more optical connections, one or more wireless connections (e.g., RF, IR, etc.), or some combination thereof. In some implementations, the buss 110 may include a serial bus, or a parallel bus, or both.

In some implementations, the main device 200 may include a watch face. More specifically, the main device 200 may include a smartwatch face that is configured to provide smartwatch features. However, the main device 200 can also include an analog watch face including interface electronics while not necessarily providing smartwatch features. Additionally, the main device 200 may include any other computing device configured to provide functionality for a user.

In some implementations, the band 300 may include a watch band that can be communicatively and/or mechanically coupled to the main device 200. In some implementations, the band 300 can be any type of wrist worn device configured to be communicatively and/or mechanically coupled to the main device 200. In some implementations, the band 300 can be worn on other parts of a user's body, such as around a leg, arm, neck, finger, etc.

Components of the main device 200 and band 300 are described in further detail below.

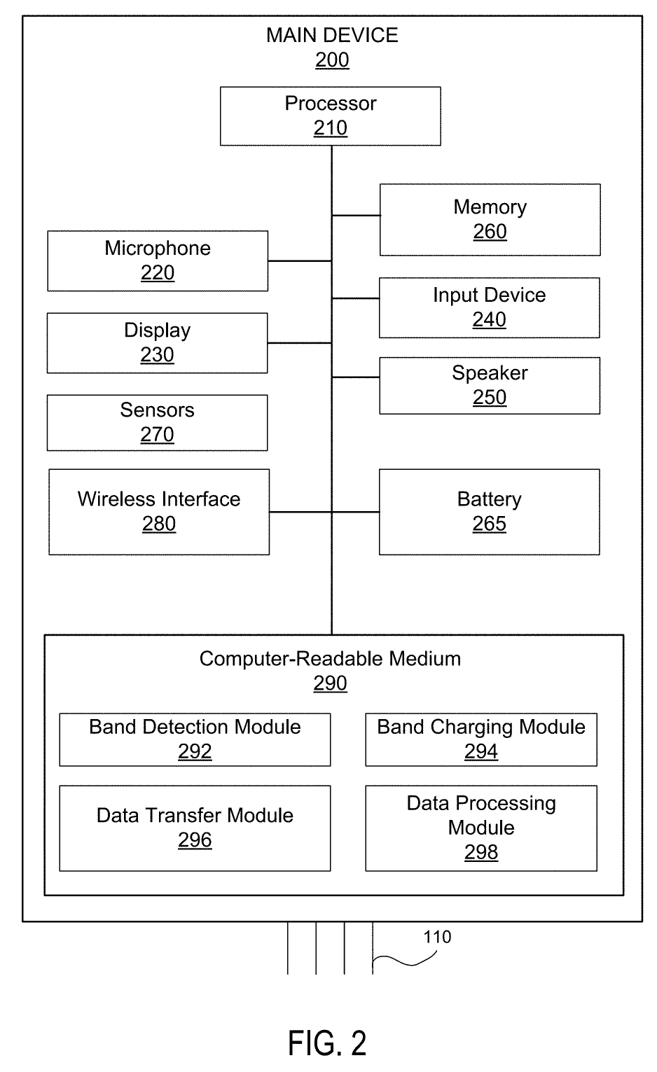

FIG. 2 illustrates a simplified block diagram of a main device 200 that may incorporate one or more implementations. Main device 200 may include a processor 210, microphone 220, display 230, input device 240, speaker 250, memory 260, battery 265, sensors 270, wireless interface 280, and computer-readable medium 290.

Processor 210 may include one or more general-purpose processors operable to carry out instructions on the main device 200. The processor 210 is coupled to other units of the main device 200 including microphone 220, display 230, input device 240, speaker 250, memory 260, battery 265, sensors 270, wireless interface 280, and computer-readable medium 290.

Microphone 220 may include one or more acoustic-to-electric transducers or sensors that converts sound into an electrical signal. The microphone 220 may provide functionality for a user of the main device 200 to record audio or issue voice commands for the main device 200. For example, the microphone 220 may be used by the user to input voice commands to execute certain smartwatch features on the main device 200.

Display 230 may include a device that displays information to a user. Examples may include an LCD screen, CRT monitor, seven-segment display, LED array, or a monolithic OLED screen. For example, the display 230 may display the time, application information, etc. for the user. The display can also be operable to provide haptic feedback to the user.

Input device 240 may include a device that accepts input from a user. Examples may include a keyboard, keypad, touchscreen, touch wheel, buttons, touchpad, or mouse. In some implementations, the microphone 220 may also function as an input device 240 for voice commands. In some implementations, a “digital crown” or other buttons attached to the main device 200 may function as input devices.

Speaker 250 may include a device that outputs sound to a user. Examples may include a built-in speaker or any other device that produces sound in response to an electrical audio signal and/or ultrasonic signal(s). The speaker 250 may be used to provide audible feedback to the user.

Memory 260 may include any magnetic, electronic, or optical memory. It can be appreciated that memory 260 may include any number of memory modules. An example of memory 260 may include dynamic random access memory (DRAM). In some implementations, memory 260 may be used to store various data that can be analyzed for sleep tracking functions.

Battery 265 can be any type of battery, energy harvesting device, or energy storage medium (e.g., fuel cell or super capacitor) used for powering the various components and functions of the main device 200. In some implementations, the battery 265 may include a lithium polymer battery.

Sensors 270 may include one or more sensors configured to obtain data accessible by the processor. The sensors 270 may also be physically coupled to the outer body of the main device 200. The plurality of sensors 270 may include, but is not limited to, an accelerometer, gyroscope, magnetometer, barometric pressure sensor, ambient temperature sensor, heart rate monitor, oximetry sensor, skin conductance sensor, skin temperature sensor, galvanic skin response sensor, ambient light sensor, etc.

Wireless interface 280 may include an interface configured to wirelessly communicate with another device. The wireless interface 280 may include Bluetooth, NFC, Wi-Fi, ZigBee, etc. In some implementations, the wireless interface 280 may facilitate wireless communication between the main device 200 and the band 300. In some implementations, the wireless interface 280 may facilitate wireless communication between the main device 200 and another device.

Computer-readable medium 290 may include a magnetic, an electronic, an optical, or other computer-readable storage medium. In this example implementation, computer-readable medium 290 includes band detection module 292, band charging module 294, data transfer module 296, and data processing module 298.

Band detection module 292 may be configured to, when executed by processor 210, detect when a band 300 communicatively and/or mechanically connects to the main device 200. As described above, the band 300 may communicatively and mechanically connect to the main device. For example, a watchstrap (e.g., band 300) may be fitted to each side of a watch face (e.g., main device 200). When the band 300 is mechanically connected to the main device 200, the band 300 may also be communicatively connected to the main device 200 by virtue of electrical connectors fitted to both the band 300 and the main device 200. The electrical connectors may facilitate data transfer between the band 300 and the main device 200 over an electrical bus (see FIG. 1). The band detection module 292 may, via processor 210, monitor the bus to determine when the band 300 is connected to the main device 200. For example, upon the band 300 being communicatively connected to the main device 200, the band detection module 292 may notify other components within the main device 200 that the band 300 has been communicatively connected to the main device 200. In some implementations, upon the band detection module 292 detecting that the band 300 has communicatively and/or mechanically connected to the main device 200, the band detection module 292 may provide a signal to the processor indicating such. In turn, the processor may affect a display, a speaker, or a haptic mechanism of the main device 200 or band 300 (e.g. via haptic feedback device 380).

In some implementations, the band detection module 292 may also detect when the band 300 has been wirelessly connected to the main device 200. The band detection module 292 may interface with the wireless interface 280 to make this determination. For example, once the wireless interface 280 establishes a wireless connection with the band 300, the band detection module 292 may notify other components within the main device 200 that the band 300 has been wirelessly connected to the main device 200.

In some implementations, the band detection module 292 may also detect when the band 300 has communicatively and/or mechanically disconnected from the main device 200. Upon the band detection module 292 detecting that the band 300 has communicatively and/or mechanically disconnected from the main device 200, the band detection module 292 may provide a signal to the processor indicating such. In turn, the processor may affect a display, a speaker, or a haptic mechanism of the main device 200 or band 300 (e.g., via haptic feedback device 380).

Band charging module 294 is configured to, when executed by processor 210, to charge a battery of the band 300. The band charging module 294 may interface with the battery 265 of the main device 200 to, in turn, charge the battery of the band 300 when the band is communicatively and mechanically connected to the band 300. For example, prior to retiring for the night, a user may communicatively and mechanically disconnect the band 300 from the main device 200. The user may then wear the band 300 throughout the night for sleep tracking purposes while connecting the main device 200 to a charger to charge throughout the night. In the morning, the battery of the band 300 may be at least partially depleted. However, once the band 300 is communicatively and mechanically connected back to the main device 200, the main device 200 may charge the battery of the band 300, since the main device 200 may ideally have a full battery charge in the morning. In some implementations, the band charging module 294 may receive an indication from the band detection module 292 when the band 300 is communicatively and mechanically connected to the main device 200. Upon receiving the indication from the band detection module 292, the band charging module 294 may interface with the battery 265 of the main device to charge the battery of the band 300 via the electrical connection between the band 300 and the main device 200.

In some implementations, band charging module 294 is configured to monitor a status of the battery of the band 300. The band charging module 294 may be configured to determine a real time status of the battery (e.g., current charge percentage, etc.). In some implementations, upon the band charging module 294 determining a state of the battery of the band 300, the band charging module 294 may provide a signal to the processor indicating such. In turn, the processor may affect a display, a speaker, or a haptic mechanism of the main device 200 or band 300.

Data transfer module 296 is configured to, when executed by processor 110, transfer data from the band 300 to the main device 200, upon the band 300 being communicatively and mechanically connected to the main device 200. The data transferred from the band 300 may be stored on a memory within the band 300. The data may have been obtained from one or more sensors within the band 300 and then stored in the memory within the band 300. For example, when the user wears the band 300 without the main device 200 during the night, the sensors on the band 300 may collect data over time. The collected data may be stored in a memory of the band 300. Upon the band 300 connecting communicatively and mechanically to the main device 200, the data transfer module 296 may facilitate, via processor 210 and the electrical connections, transfer of the data from the memory within the band 300 to the memory 260 within the main device 200. The data transfer module 296 may facilitate the transfer of the data upon receiving an indication from the band detection module 292 that the band 300 has been communicatively connected to the main device 200.

In some implementations, the data transfer module 296 may also facilitate transfer of data from the band 300 to the main device 200 via the wireless interface 280. For example, upon the band 300 and the main device 200 establishing a wireless connection, the band detection module 292 may send an indication to the data transfer module 296 indicating that band 300 and main device 200 have established a wireless connection. The data transfer module 296 may then facilitate a wireless transfer of the data from the band 300 to the main device 200.

Data processing module 298 may be configured to, when executed by processor 110, process the data transferred to the main device 200 from the band, by the data transfer module 296. The data processing module 298 may process the data that is transferred into the memory 260. In some implementations, the data may include data obtained by one or more sensors present within the band 300. The data may include, in one example, data captured by an accelerometer and heart rate monitor. The data processing module 298 may facilitate the processing of the accelerometer data and heart rate monitor data in order to determine the “quality” of a user's sleep (e.g., sleep tracking). In some implementations, the data processing module 298 may comprise one or more algorithms for processing the data stored in the memory 260. The processed data may be displayed to the user via the display 230.

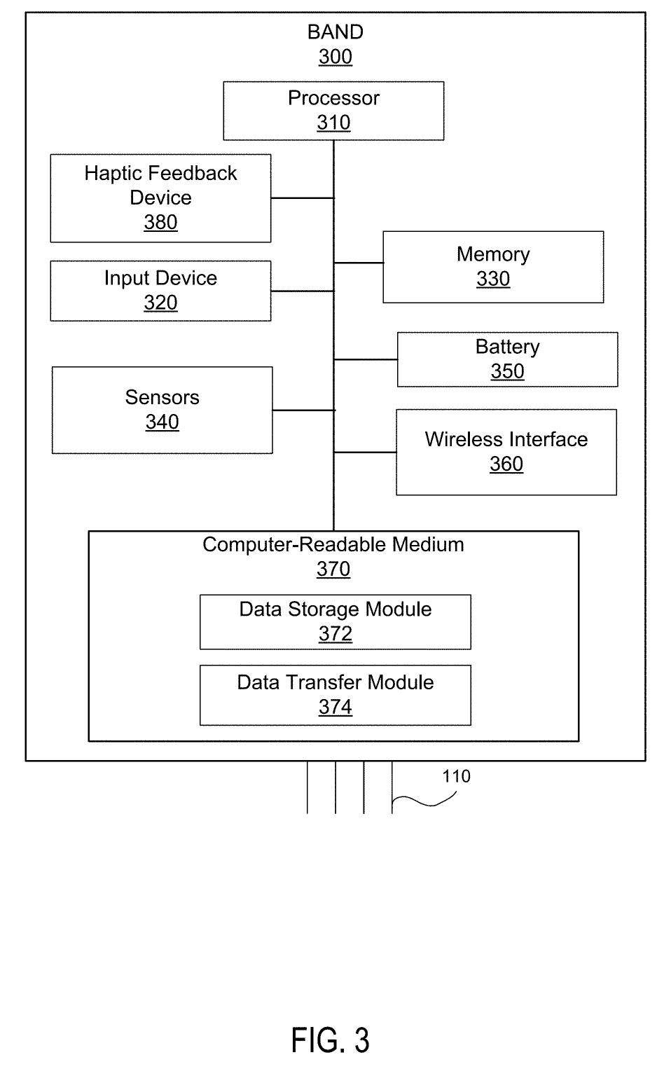

FIG. 3 illustrates a simplified block diagram of a band 300 that may incorporate one or more implementations. The band 300 may include a processor 310, input device 320, memory 330, sensors 340 and battery 350.

In some implementations, band 300 may include a watchband that could be worn around a user's wrist. However, the band 300 can include a type of band that may be worn around some portion of a user, e.g., a user extremity. The exterior of the band 300 may be composed of materials such as metal, leather, rubber, or other material that may be useful for a watchband. As described above, the band 300 may be configured to mechanically and communicatively connecting to the main device 200. In other words, the band 300 may be detachable from the main device 200. A main device 200 may be associated with one or more bands 300.

Processor 310 may include one or more general-purpose processors operable to carry out instructions on the band 300. The processor 310 is coupled to other units of the band 300 including input device 320, memory 330, sensors 340, battery 350, wireless interface 360, and computer-readable medium 370. The processor 310 in the band 300 may include a low power processor.

Input device 320 may include one or more devices that accepts input from a user. Examples may include a keyboard, keypad, or mouse. In some implementations, the input device 320 can be one or more buttons positioned on a side(s) of the band 300. In some implementations, the buttons positioned on the side can have one or more functions during a regular operation where the band 300 and main device 200 are connected, and different functions when the band 300 is disconnected from the main device 200.

Memory 330 may include a magnetic memory, an electronic memory, an optical memory, or a combination thereof, just to name a few examples. It can be appreciated that memory 330 may include any number of memory modules. An example of memory 330 may include dynamic random access memory (DRAM). In some implementations, memory 330 may be used to store various data obtained by the sensors 340.

Sensors 340 may include one or more sensors configured to obtain data accessible by the processor. The sensors 340 may be physically coupled to the outer body of the band 300 or may be disposed within the band 300. The plurality of sensors 340 may include, but is not limited to, an accelerometer, gyroscope, magnetometer, barometric pressure sensor, ambient temperature sensor, heart rate monitor, oximetry sensor, skin conductance sensor, skin temperature sensor, galvanic skin response sensor, ambient light sensor, etc. In some implementations, at least one of the sensors 340 part of the band 300 may be different than at least one of the sensors 270 part of the main device 200. In other words, there may not be a comprehensive overlap between the sensors of the band 300 and the main device 200. Since certain sensors can be disposed within the band 300, it may be redundant to duplicate those sensors in the main device 200.

Battery 350 may represent one or more of a battery, an energy harvesting device, an energy storage medium (e.g., fuel cell or super capacitor), or the like or some combination thereof that may be used for powering all or part of the various components and functions of the band 300. In some implementations, the battery 350 may include a lithium polymer battery. In some implementations, the battery 350 may include a supercapacitor. In some implementations, the battery 350 of the band 300 may be configured to be charged by the battery 265 of the main device 200.

Wireless interface 360 may include one or more interfaces configured to wirelessly communicate with another device. The wireless interface 360 may include Bluetooth, NFC, Wi-Fi, ZigBee, etc. In some implementations, the wireless interface 360 may facilitate wireless communication between the band 30 and the main device 200. For example, wireless interface 360 may initiate and maintain a wireless connection with wireless interface 280 of main device 200. In some implementations, the wireless interface 360 may facilitate wireless communication between the band 300 and another device.

Computer-readable medium 370 may include one or more of a magnetic, an electronic, an optical, or other computer-readable storage medium. Computer-readable medium 370 includes data storage module 372 and data transfer module 374.

Data storage module 372 is configured to, when executed by processor 110, store data obtained by the one or more sensors 340 into the memory 330. As described above, the one or more sensors 340 may obtain data that may be pertinent to tracking various activities associated with the user. For example, the one or more sensors 340 may obtain data that can be used for determining the quality of a user's sleep (e.g., sleep tracking). Upon the one or more sensors 340 obtaining sensor data, the data storage module 372 may, via processor 310, facilitate the recording of the obtained sensor data into the memory 330. In some implementations, all or part of the data may be compressed or processed prior to being stored into the memory 330. The data storage module 372 may also facilitate the initiation of the sensors 340 to begin obtaining sensor data upon (or at a specific time afterward of) the band 300 being disconnected from the main device 200.

Data transfer module 374 is configured to, when executed by processor 110, facilitate transfer of the sensor data stored in the memory 330 to the main device 200. The data transfer module 374 may facilitate the transfer of the sensor data stored in the memory 330 to the main device 200 upon the band 300 being electrically connected to the main device 200. In some implementations, the data transfer module 374 may interface with the data transfer module 296 of the main device 200 to facilitate the transfer of the sensor data. For example, when the band 300 and the main device 200 are communicatively connected to each other, the band detection module 292 (FIG. 2) may also send an indication to both the data transfer module 296 of the main device 200 and the data transfer module 374 of the band 300. Upon receiving the indication, both the data transfer module 296 of the main device 200 and the data transfer module 374 of the band 300 may interface with one another to facilitate the transfer of the data from the data stored in the memory 330 of the band 300 to the main device 200. As described above, upon receiving the sensor data, the main device 200 may store and process the sensor data.

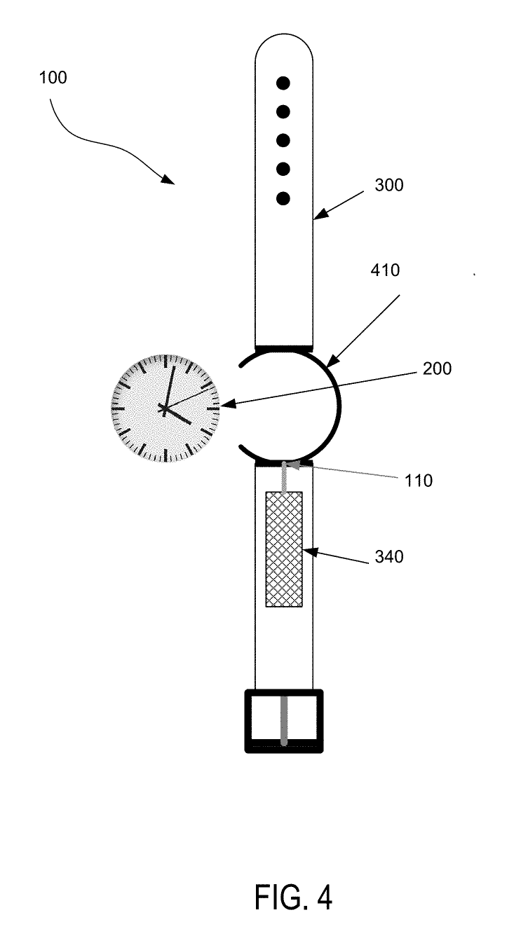

FIG. 4 depicts a system 100 including a main device 200 and a band 300 according to some implementations. In addition to the main device 200 and the band 300, the system 100 also includes frame 410, bus 110, and sensors 340. In some implementations, the system 100 is a smartwatch.

Some, possibly a majority, of the smartwatch features may be provided by the main device 200. For example, the main device 200 may include at least a processor, memory, and display. The main device 200 may provide many smartwatch functionalities to the user. For example, the main device 200 may include a computerized wristwatch with functionality that is enhanced beyond basic timekeeping. These functionalities may include calculations, translations, game-playing, and execution of mobile applications using a mobile operating system. Additional functionalities may include the ability to function as portable a media player, offering playback of radio, audio, and video files to the user via a Bluetooth or USB headset. The main device 200 may also feature full mobile phone capabilities, such as the ability to make and answer phone calls. The main device 200 may have a rechargeable battery and a graphical display, which may function as a touch screen. Additionally, the main device may also include or interface with peripheral devices such as a camera, thermometer, accelerometer, altimeter, barometer, compass, GPS receiver, speaker and SDcard that is recognized as a mass storage device by a computer. Software configured to be executed on the main device 200 may include a map display, scheduler and personal organizer, calculator, and various kinds of digital watch faces. The main device 200 may communicate with external devices such as sensors, a wireless headset, or a heads-up display.

Similar to computers, the main device 200 may collect information from internal or external sensors. The main device 200 may control, or retrieve data from, other instruments or computers. The main device 200 may support wireless technologies like Bluetooth, Wi-Fi, and GPS. Thus, as mentioned above, the main device 200 may be operable to provide many primary functionalities for a user. However, the main device 200 may require nightly charging to ensure that the battery of the main device 200 has enough charge to last throughout the day. Functionalities such as sleep tracking may require the user to wear the device throughout the night such that sensors may capture sensor data that can be used for the sleep tracking functions. In addition to the requirement for nightly charging, wearing the main device 200 (e.g., smartwatch) can be uncomfortable for a user. Often times, the face of the main device 200 may get caught under a pillow, bang against a headboard, or just be plain uncomfortable for a user to wear throughout the night.

Accordingly, the system 100 allows for the band 300 to be separated (electrically and mechanically) from the main device 200. The band 300 may contain various electronics that can independently operate without the band 300 having to be connected to the main device 200. The band 300 may include various sensors that can obtain sensor data while the band is worn around a user's wrist (or other extremity) even when the main device 200 is not electrically and mechanically connected to the band 300. The band 300 may also include a battery, memory, and other electronics.

The system also includes a frame 410 operable to hold the main device 200. The main device 200 may be “snapped” into the frame 410 when the user wishes to use both the main device 200 and the band 300 together. The frame 410 may be designed to be lightweight and unobtrusive to the user such that the user can wear the band 300 and the frame 410 without discomfort throughout the night. In some implementations, the frame 410 may also be removable from the band 300 and the ends of the band may be connected together to form a closed loop around the user's wrist. For example, the ends of the band 300 may be coupled to one or more magnets operable to join the two ends of the band 300 together to form the closed loop. In some implementations, the frame 410 may have a “cutout” area such that when the main device 200 is snapped into the frame 410, the bus 110 of the band 300 may communicatively connect to the bus of the main device 200. In some implementations, the band 300 and the main device 200 may communicate with each other wirelessly.

In an illustrative example, a user may wear the system 100 (e.g., combination of connected main device 200 and band 300) throughout the day. The user may user various functions offered by the main device 200 throughout the user's day. At night, before the user is about to retire to bed, the user may disconnect (communicatively and mechanically) the main device 200 from the band 300. The user may then place the main device 200 in a charging cradle or connect it to a charger, such that the battery of the main device 200 that may have been partially or fully depleted throughout the day may be restored to full charge overnight. The user may also continue to wear the band 300 around the user's wrist throughout the rest of the night. Since the band 300 includes its own battery, the band 300 may continue to provide functionality throughout the night. Upon the user disconnecting the main device 200 from the band 300, the band may begin to start obtaining data from the one or more sensors within the band, in a process similar to the one described with respect to FIG. 3. In the context of sleep tracking, the sensors 340 within the band 300 may collect sensor data pertinent to analyzing the user's sleep patterns. For example, an accelerometer, microphone, and/or heart rate sensor can capture sensor data while the user is wearing the band 300 during the user's sleep. In some implementations, the sensors 340 may include low-power sensors. In some implementations, the band 300 may include one or more buttons that the user can press when the user wishes to begin the sleep tracking functionality. The data captured by the sensors 340 during the user's sleep can be stored in the memory within the band 300 (e.g., via the data storage module 372).

Upon the user waking the next morning, the user may reconnect the band 300 to the main device 200. At this time, the battery of the main device 200 may have been fully charged overnight. Upon the band 300 being electrically and mechanically connected to the main device 200, the band 300 may transfer the sensor data stored in the memory to the main device 200 (e.g., via data transfer module 374 and data transfer module 296 of the main device 200). Additionally, the main device's 200 battery may charge (via band charging module 294) the battery of the band 300 using the battery of the main device 200, since the battery of the band 300 may have been partially depleted overnight.

In some implementations, the sensors 340 on the band 300 may only need to sample data every 30 seconds in order to obtain data useful for sleep tracking. In an example, ten hours of obtained sensor data may only deplete 2 mA-hours of the band's 300 battery. The band's 300 battery can be a small 50 mA-hour battery that can be appropriately sized to fit within the band 300. Thus, the main device's 200 battery would not be depleted a significant amount when charging the band's 300 battery in the morning.

Additionally, upon the band 300 being communicatively and mechanically connected to the main device 200, and after the band 300 has transferred the sensor data stored in its memory to the main device 200, the main device 200 may process the transferred sensor data to provide the user with analytical data that the user can view on the main device's 200 display. For example, the main device 200 may display sleep patterns and various other sleep tracking metrics based on the sensor data.

In another example, prior to going for a swim in water, the user may disconnect the band 300 from the main device 200. Since the main device 200 may contain sensitive electronics, the user may wish to not submerge the main device 200 underwater. However, the band 300 may be designed to be water resistant or waterproof such that the sensors, battery, memory, and processor within the band 300 may not be susceptible to water damage. The various sensors 340 within the band 300 may continue to track the user's activity while the user is swimming. For example, a gyroscope, accelerometer, and heart rate sensor within the band 300 may continue to obtain sensor data. The obtained sensor data may be stored within the band's 300 memory. Upon the user reconnecting the band 300 to the main device 200, the band 300 may transfer the stored data to the main device 200 (or the main device 200 may obtain the stored data from the band 300) in a manner similar to the one described in the previous example with respect to sleep tracking. In some implementations, upon the stored data (or a portion of the stored data) being transferred to or obtained by the main device 200, the data transfer module 296 may provide a signal to the processor indicating such. In turn, the processor may affect a display, a speaker, or a haptic mechanism of the main device 200 or band 300.

The main device 200 may then process the sensor data and display analytics pertaining the user's swim. For example, the main device 200 may display the amount of calories burned, distance swum, total number of laps, number of strokes, etc. pertaining to the swim and the user's average heart rate. In some implementations, upon the stored data (or a portion of the stored data) being processed by the main device 200, the processor of the main device 200 may affect a display, a speaker, or a haptic mechanism of the main device 200 or band 300.

Thus, it can be appreciated that the band 300 may be detached from the main device 200 in various situations beyond sleep tracking and swimming, where the band 300 can continue to obtain sensor data via the sensors 340.

In some implementations, upon the band 300 being reconnected to the main device 200, the main device 200, upon receiving the sensor data from the band 300, may transmit (either wirelessly or via an electrical connection) the sensor data to another device for further processing. For example, the main device 200 may transmit the sensor data to a cloud service or desktop computer for further processing. Alternatively, the main device 200 may first process the data locally prior to transferring the processed data to another device or cloud service.

It can be appreciated that the band 300 may be interchangeable and replaceable. Thus, one day the user can use a red colored leather band 300 and the next day the user could use a brushed aluminum metal band 300. The user may have access to various bands 300 depending on the user's personal preferences. Each band may include the components described with respect to FIG. 3.

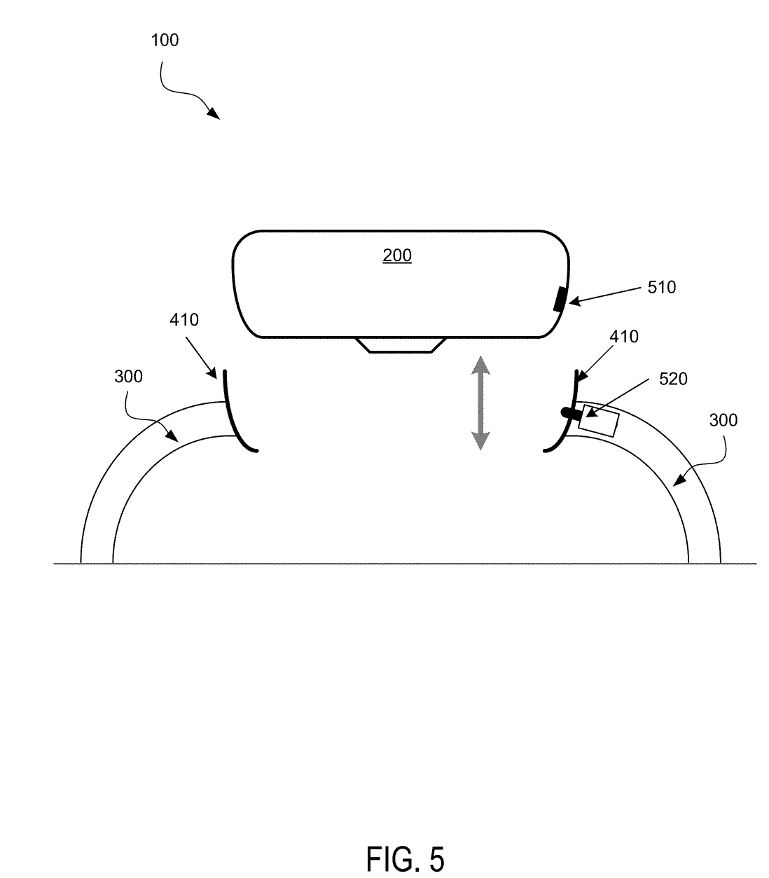

FIG. 5 illustrates a main device 200 configured to be communicatively and mechanically connected to a band 300. The band 300 may include a frame 410, a contact pad 510, and a contact pins 520. The main device 200 may “snap in” to the band 300 by being pressed into the frame. The main device 200 may also snap in to the band from the bottom or side. The frame 410 may be operable to securely hold the main device 200 in place such that the user can wear the entire smartwatch around the user's wrist by securing the band 300 around the user's wrist. Additionally, the frame 410 may include a contact pins 520. The contact pins 520 may be coupled the bus 110 within the band 300. The contact pins 520 may be operable to create an electrical connection between the bus within the band 300 and the bus within the main device 200. The main device 200 may include a contact pad 510 disposed within the device. The contact pad 510 may make an electrical contact with the contact pins 520 when the main device 200 is snapped into the frame 410. The contact pins 520 may be spring loaded such that when the main device 200 is snapped into the frame 410, the body of the main device 200 may compress the contact pins 520 until the main device 200 slides down the frame 410 enough such that the contact pins 520 can “pop” into the contact pad 510. The contact pad 510 may be coupled to the bus within the main device 200, such that when the contact pad 510 and contact pins 520 make contact, an electrical connection between the bus of the main device 200 and the bus of the band 300 may be completed.

While a single contact pad 510 and contact pins 520 are shown in the figure, a contact pad and contact pin may be placed on each end of the main device 200 and smart band 300.

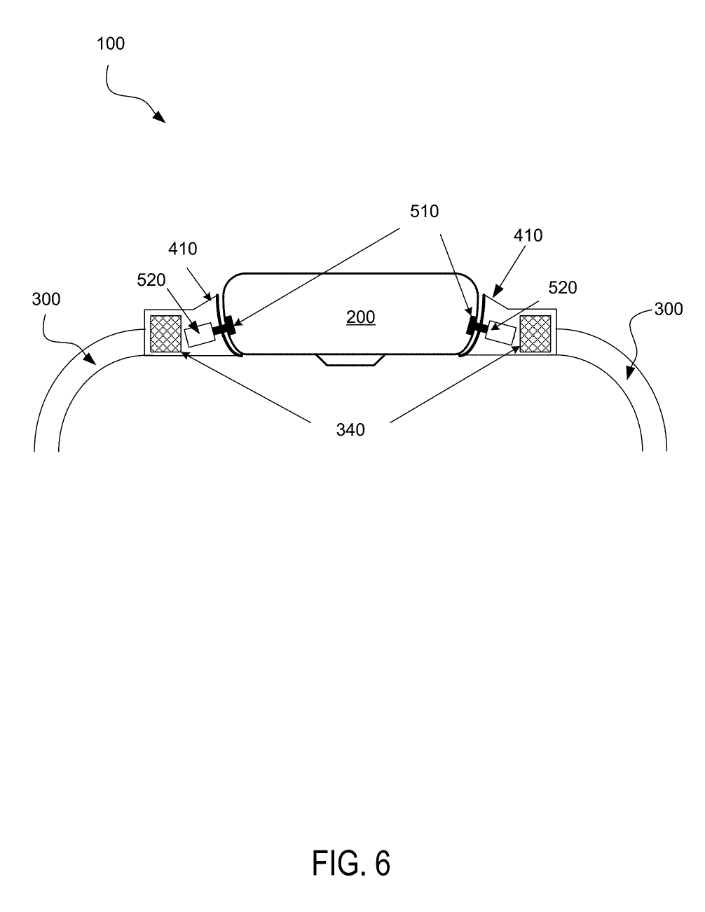

FIG. 6 illustrates a main device 200 communicatively and mechanically connected to a band 300. The figure illustrates the main device 200 fully “snapped” into the band 300. The main device 200 is being securely held by the frame 410 of the band. Further, each side of the band 300 includes the contact pins 520 within the frame 410. As shown here, when the main device 200 is fully snapped into the frame 410 of the band 300, the contact pins 520 makes an electrical contact with the contact pad 510 disposed on the main device 200. Accordingly, busses within each of the main device 200 and band 300 may be electrically connected with one another (by virtue of the contact pins 520 and contact pad 510) when the main device 200 is snapped into the band 300.

In some implementations, the sensors 340 may be placed within the frame 410 instead of within the band 300. Placing the sensors 340 within the frame 410 may allow the placing of the sensors 340 within a rigid enclosure. By placing the sensors within the rigid frame 410, the sensors may not need to be designed with flexible cabling for the band 300. The battery of the band may fit on either side of the frame 410, and the sensors 430 may be small enough to fit within the frame 410.

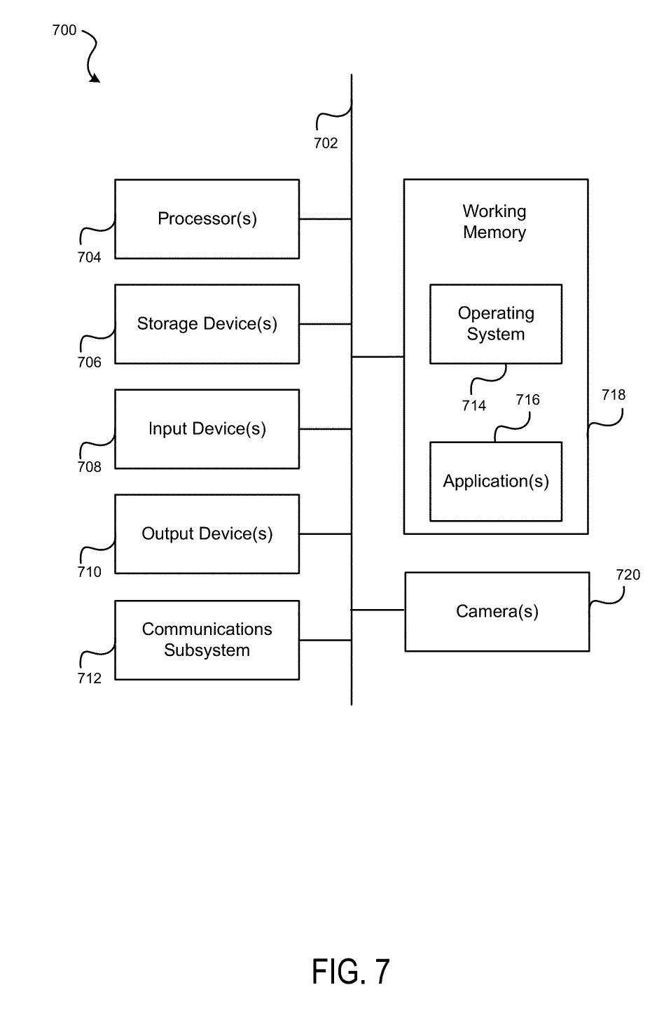

FIG. 7 illustrates an example of a computing system in which one or more embodiments may be implemented. All or part of computer system as illustrated in FIG. 7 may be incorporated as part of the above described computerized device. For example, computer system 700 can represent some of the components of a smartwatch, television, a computing device, a server, a desktop, a workstation, a control or interaction system in an automobile, a tablet, a netbook or other suitable computing system. A computing device may include a computing device with an image capture device or input sensory unit and a user output device. An image capture device or input sensory unit may include a camera device. A user output device may include a display unit. Examples of a computing device include but are not limited to video game consoles, tablets, smart phones and other hand-held devices. FIG. 7 provides a schematic illustration of one embodiment of a computer system 700 that can perform the methods provided by various other embodiments, as described herein, and/or can function as the host computer system, a remote kiosk/terminal, a point-of-sale device, a telephonic or navigation or multimedia interface in an automobile, a computing device, a set-top box, a table computer and/or a computer system. FIG. 7 is meant only to provide a generalized illustration of various components, any or all of which may be utilized as appropriate. FIG. 7, therefore, broadly illustrates how individual system elements may be implemented in a relatively separated or relatively more integrated manner. In some embodiments, elements of computer system 700 may be used to implement functionality of the system 100 in FIG. 1.

The computer system 700 is shown comprising hardware elements that can be electrically coupled via a bus 702 (or may otherwise be in communication, as appropriate). The hardware elements may include one or more processors 704, including without limitation one or more general-purpose processors and/or one or more special-purpose processors (such as digital signal processing chips, graphics acceleration processors, and/or the like); one or more input devices 708, which can include without limitation one or more cameras, sensors, a mouse, a keyboard, a microphone configured to detect ultrasound or other sounds, and/or the like; and one or more output devices 710, which can include without limitation a display unit such as the device used in some implementations, a printer and/or the like.

In some implementations, various input devices 708 and output devices 710 may be embedded into interfaces such as display devices, tables, floors, walls, and window screens. Furthermore, input devices 708 and output devices 710 coupled to the processors may form multi-dimensional tracking systems.

The computer system 700 may further include (and/or be in communication with) one or more non-transitory storage devices 706, which can comprise, without limitation, local and/or network accessible storage, and/or can include, without limitation, a disk drive, a drive array, an optical storage device, a solid-state storage device such as a random access memory (“RAM”) and/or a read-only memory (“ROM”), which can be programmable, flash-updateable and/or the like. Such storage devices may be configured to implement any appropriate data storage, including without limitation, various file systems, database structures, and/or the like.

The computer system 700 might also include a communications subsystem 712, which can include without limitation a modem, a network card (wireless or wired), an infrared communication device, a wireless communication device and/or chipset (such as a Bluetooth™ device, an 802.11 device, a Wi-Fi device, a WiMax device, cellular communication facilities, etc.), and/or the like. The communications subsystem 712 may permit data to be exchanged with a network, other computer systems, and/or any other devices described herein. In many embodiments, the computer system 700 will further comprise a non-transitory working memory 718, which can include a RAM or ROM device, as described above.

The computer system 700 also can comprise software elements, shown as being currently located within the working memory 718, including an operating system 714, device drivers, executable libraries, and/or other code, such as one or more application programs 716, which may comprise computer programs provided by various embodiments, and/or may be designed to implement methods, and/or configure systems, provided by other embodiments, as described herein. Merely by way of example, one or more procedures described with respect to the method(s) discussed above might be implemented as code and/or instructions executable by a computer (and/or a processor within a computer); in an aspect, then, such code and/or instructions can be used to configure and/or adapt a general purpose computer (or other device) to perform one or more operations in accordance with the described methods.

A set of these instructions and/or code might be stored on a computer-readable storage medium, such as the storage device(s) 706 described above. In some cases, the storage medium might be incorporated within a computer system, such as computer system 700. In other embodiments, the storage medium might be separate from a computer system (e.g., a removable medium, such as a compact disc), and/or provided in an installation package, such that the storage medium can be used to program, configure and/or adapt a general purpose computer with the instructions/code stored thereon. These instructions might take the form of executable code, which is executable by the computer system 700 and/or might take the form of source and/or installable code, which, upon compilation and/or installation on the computer system 700 (e.g., using any of a variety of generally available compilers, installation programs, compression/decompression utilities, etc.) then takes the form of executable code.

Substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed. In some embodiments, one or more elements of the computer system 700 may be omitted or may be implemented separate from the illustrated system. For example, the processor 704 and/or other elements may be implemented separate from the input device 708. In one embodiment, the processor is configured to receive images from one or more cameras that are separately implemented. In some embodiments, elements in addition to those illustrated in FIG. 7 may be included in the computer system 700.

Some embodiments may employ a computer system (such as the computer system 700) to perform methods in accordance with the disclosure. For example, some or all of the procedures of the described methods may be performed by the computer system 700 in response to processor 704 executing one or more sequences of one or more instructions (which might be incorporated into the operating system 714 and/or other code, such as an application program 716) contained in the working memory 718. Such instructions may be read into the working memory 718 from another computer-readable medium, such as one or more of the storage device(s) 706. Merely by way of example, execution of the sequences of instructions contained in the working memory 718 might cause the processor(s) 704 to perform one or more procedures of the methods described herein.

The terms “machine-readable medium” and “computer-readable medium,” as used herein, refer to any medium that participates in providing data that causes a machine to operate in a specific fashion. In some embodiments implemented using the computer system 700, various computer-readable media might be involved in providing instructions/code to processor(s) 704 for execution and/or might be used to store and/or carry such instructions/code (e.g., as signals). In many implementations, a computer-readable medium is a physical and/or tangible storage medium. Such a medium may take many forms, including but not limited to, non-volatile media, volatile media, and transmission media. Non-volatile media include, for example, optical and/or magnetic disks, such as the storage device(s) 706. Volatile media include, without limitation, dynamic memory, such as the working memory 718. Transmission media include, without limitation, coaxial cables, copper wire and fiber optics, including the wires that comprise the bus 702, as well as the various components of the communications subsystem 712 (and/or the media by which the communications subsystem 712 provides communication with other devices). Hence, transmission media can also take the form of waves (including without limitation radio, acoustic and/or light waves, such as those generated during radio-wave and infrared data communications).

Common forms of physical and/or tangible computer-readable media include, for example, a floppy disk, a flexible disk, hard disk, magnetic tape, or any other magnetic medium, a CD-ROM, any other optical medium, punchcards, papertape, any other physical medium with patterns of holes, a RAM, a PROM, EPROM, a FLASH-EPROM, any other memory chip or cartridge, a carrier wave as described hereinafter, or any other medium from which a computer can read instructions and/or code.

Various forms of computer-readable media may be involved in carrying one or more sequences of one or more instructions to the processor(s) 1004 for execution. Merely by way of example, the instructions may initially be carried on a magnetic disk and/or optical disc of a remote computer. A remote computer might load the instructions into its dynamic memory and send the instructions as signals over a transmission medium to be received and/or executed by the computer system 700. These signals, which might be in the form of electromagnetic signals, acoustic signals, optical signals and/or the like, are all examples of carrier waves on which instructions can be encoded, in accordance with various implementations.

The communications subsystem 712 (and/or components thereof) generally will receive the signals, and the bus 702 then might carry the signals (and/or the data, instructions, etc. carried by the signals) to the working memory 718, from which the processor(s) 704 retrieves and executes the instructions. The instructions received by the working memory 718 may optionally be stored on a non-transitory storage device 706 either before or after execution by the processor(s) 704.

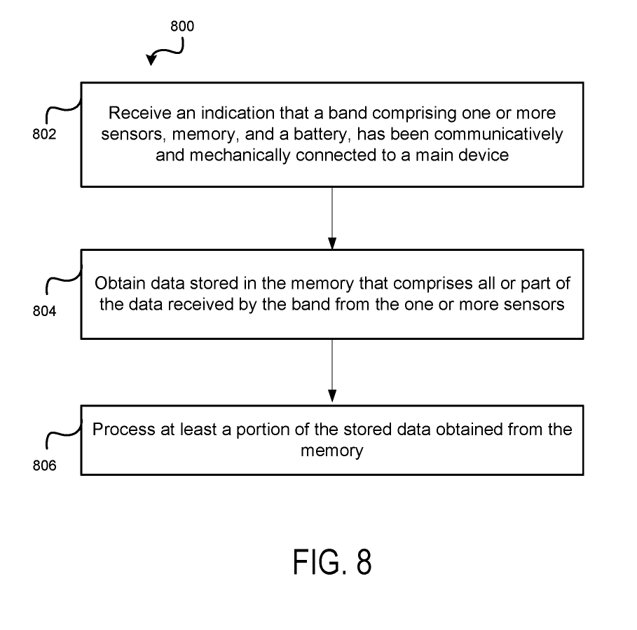

With the various teachings presented herein in mind, attention is drawn next to FIG. 8, which is flow-diagram illustrating an example process 800 that may be implemented in a system 100, and in certain implementations, a main device 200, for example, as illustrated in FIG. 1, in accordance with certain implementations.

For example, at block 802, one or more indications may be received or otherwise detected that a band 300, e.g., possibly comprising one or more sensors, memory, and a battery, has been communicatively and mechanically connected to a main device 200. At block 804, for example, in response to one or more of the indications at block 802, the main device 200 (e.g., a processor of the main device) may obtain data stored in the memory of the band that comprises all or part of the data received by the band from the one or more sensors, or otherwise corresponding to such one or more sensors. At block 806, for example, main device 200 (e.g., a processor of the main device) may process in some manner all or part of the stored data obtained from the memory.

The methods, systems, and devices discussed above are examples. Various configurations may omit, substitute, or add various procedures or components as appropriate. For instance, in alternative configurations, the methods may be performed in an order different from that described, and/or various stages may be added, omitted, and/or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Also, configurations may be described as a process which is depicted as a flow diagram or block diagram. Although each may describe the operations as a sequential process, many of the operations can be performed in parallel or concurrently. In addition, the order of the operations may be rearranged. A process may have additional steps not included in the figure. Furthermore, examples of the methods may be implemented by hardware, software, firmware, middleware, microcode, hardware description languages, or any combination thereof. When implemented in software, firmware, middleware, or microcode, the program code or code segments to perform the necessary tasks may be stored in a non-transitory computer-readable medium such as a storage medium. Processors may perform the described tasks.

Having described several example configurations, various modifications, alternative constructions, and equivalents may be used without departing from the spirit of the disclosure. For example, the above elements may be components of a larger system, wherein other rules may take precedence over or otherwise modify the application of the implementations described herein. Also, a number of steps may be undertaken before, during, or after the above elements are considered.

What is claimed is:

1. A system, comprising:
a main device comprising a processor;
a band comprising one or more sensors, memory, and a battery, wherein the band is configured to communicatively and mechanically connect to the main device, and to store data received from the one or more sensors into the memory;
wherein, being connected to the band, the processor is configured to:
obtain the stored data from the memory of the band; and
process the stored data obtained from the memory of the band.

2. The system of claim 1, wherein, being connected to the band, the main device is further configured to charge the battery of the band.

3. The system of claim 1, wherein the main device further comprises a wireless interface, and the processor is further configured to initiate transmission of the data to another device via the wireless interface.

4. The system of claim 1, wherein the band is configured to communicatively connect to the main device via an Inter-Integrated Circuit (I²C) bus.

5. The system of claim 1, wherein the one or more sensors comprises at least one of: an accelerometer, a gyroscope, a galvanic skin response (GSR) sensor, a skin temperature sensor, an ambient temperature sensor, an ambient light sensor, a heart rate monitor, a bioelectric impedance measuring device, a barometer, or a microphone.

6. The system of claim 1, wherein the band is further configured to maintain a wireless communicative connection with the main device without being mechanically connected to the main device.

7. The system of claim 1, wherein the processor is further configured to affect at least one of:
a display;
a speaker;
a haptic mechanism;
or a light source, of the main device in response to at least one of:
(1) a determination that the main device is communicatively connected to the band;
(2) a determination that the main device is communicatively disconnected from the band;
(3) a determination that the main device is mechanically connected to the band;
(4) a determination that the main device is mechanically disconnected from the band;
(5) a determination that the processor has obtained at least a portion of the stored data from the memory of the band;
(6) a determination that main device has processed at least a portion of the stored data obtained from the memory of the band;
(7) a determination corresponding to a state of the battery of the band; or
(8) some combination of (1) through (7).

8. The system of claim 1, wherein the battery of the band has a charge capacity between 20 milliampere hour (mAh) and 100 mAh.

9. The system of claim 1, wherein the data received by the band comprises data associated with tracking properties of a user's sleep.

10. A method, comprising:
receiving an indication that a band comprising one or more sensors, memory, and a battery, has been communicatively and mechanically connected to a main device;
in response to receiving the indication that the band has been communicatively and mechanically connected to the main device:
obtaining data stored in the memory that comprises data received by the band from the one or more sensors; and
processing the stored data obtained from the memory.

11. The method of claim 10, further comprising, in response to receiving the indication that the band has been communicatively and mechanically connected to the main device, charging the battery of the band.

12. The method of claim 10, further comprising initiating transmission of the data to another device.

13. The method of claim 10, wherein the band is configured to communicatively connect to the main device via an Inter-Integrated Circuit (I²C) bus.

14. The method of claim 10, wherein the one or more sensors comprises at least one of an accelerometer, a gyroscope, a galvanic skin response (GSR) sensor, a skin temperature sensor, an ambient temperature sensor, an ambient light sensor, a heart rate monitor, a bioelectric impedance measuring device, a barometer, or a microphone.

15. The method of claim 10, wherein the band is configured to maintain a wireless communicative connection with the main without being mechanically connected to the main device.

16. The method of claim 10, further comprising affecting at least one of:
a display;
a speaker;
a haptic mechanism;
or a light source, of the main device in response to at least one of:
(1) a determination that the main device is communicatively connected to the band;
(2) a determination that the main device is communicatively disconnected from the band;
(3) a determination that the main device is mechanically connected to the band;
(4) a determination that the main device is mechanically disconnected from the band;
(5) a determination that the processor has obtained at least a portion of the stored data from the memory of the band;
(6) a determination that main device has processed at least a portion of the stored data obtained from the memory of the band;
(7) a determination corresponding to a state of the battery of the band; or
(8) some combination of (1) through (7).

17. The method of claim 10, wherein the battery of the band has a charge capacity between 20 milliampere hour (mAh) and 100 mAh.

18. The method of claim 10, wherein the data received by the band comprises data associated with tracking properties of a user's sleep.

19. An apparatus, comprising:
means for receiving an indication that a band comprising one or more sensors, memory, and a battery, has been communicatively and mechanically connected to a main device;
in response to receiving the indication that the band has been communicatively and mechanically connected to the main device:
means for obtaining data stored in the memory that comprises data received by the band from the one or more sensors; and
means for processing the stored data obtained from the memory.

20. The apparatus of claim 19, wherein, being connected to the band, the apparatus further comprises means for charging the battery of the band.

21. The apparatus of claim 19, further comprising means for initiating transmission of the data to another device.

22. The apparatus of claim 19, wherein the band is configured to communicatively connect to the main device via an Inter-Integrated Circuit (I²C) bus.

23. The apparatus of claim 19, further comprising means for maintaining a wireless communicative connection with the band without being mechanically connected to the band.

24. The apparatus of claim 19, further comprising means for affecting at least one of:
a display;
a speaker;
a haptic mechanism;
or a light source, of the main device in response to at least one of:
(1) a determination that the main device is communicatively connected to the band;
(2) a determination that the main device is communicatively disconnected from the band;
(3) a determination that the main device is mechanically connected to the band;
(4) a determination that the main device is mechanically disconnected from the band;
(5) a determination that the processor has obtained at least a portion of the stored data from the memory of the band;
(6) a determination that main device has processed at least a portion of the stored data obtained from the memory of the band;
(7) a determination corresponding to a state of the battery of the band; or
(8) some combination of (1) through (7).

25. The apparatus of claim 19, wherein the data received by the band comprises data associated with tracking properties of a user's sleep.

26. One or more non-transitory computer-readable media storing computer-executable instructions that, when executed, cause one or more computing devices included in a mobile device to:
receive an indication that a band comprising one or more sensors, memory, and a battery, has been communicatively and mechanically connected to a main device;
in response to receiving the indication that the band has been communicatively and mechanically connected to the main device:
obtain data stored in the memory that comprises data received by the band from the one or more sensors; and
process the stored data obtained from the memory.

27. The non-transitory computer-readable media of claim 26, wherein the instructions that, when executed, further cause the one or more computing devices to, in response to receiving the indication that the band has been communicatively and mechanically connected to the main device, charge the battery of the band.

28. The non-transitory computer-readable media of claim 26, wherein the instructions that, when executed, further cause the one or more computing devices to initiate transmission of the data to another device.

29. The non-transitory computer-readable media of claim 26, wherein the instructions that, when executed, further cause the one or more computing devices to affect at least one of:
a display;
a speaker;
a haptic mechanism;
or a light source, of the main device in response to at least one of:
(1) a determination that the main device is communicatively connected to the band;
(2) a determination that the main device is communicatively disconnected from the band;
(3) a determination that the main device is mechanically connected to the band;
(4) a determination that the main device is mechanically disconnected from the band;
(5) a determination that the processor has obtained at least a portion of the stored data from the memory of the band;
(6) a determination that main device has processed at least a portion of the stored data obtained from the memory of the band;
(7) a determination corresponding to a state of the battery of the band; or
(8) some combination of (1) through (7).

30. The non-transitory computer-readable media of claim 26, wherein the data received by the band comprises data associated with tracking properties of a user's sleep.
